(19) United States
(12) Patent Application Publication
(10) Pub. No.: US 2019/0091044 A1
(43) Pub. Date: Mar. 28, 2019

(54) TREATMENT METHOD

(71) Applicant: TERUMO KABUSHIKI KAISHA, Tokyo (JP)

(72) Inventor: Takeshi DOUSONO, Fujinomiya-shi (JP)

(73) Assignee: TERUMO KABUSHIKI KAISHA, Tokyo (JP)

(21) Appl. No.: 16/043,214

(22) Filed: Jul. 24, 2018

(30) Foreign Application Priority Data
Sep. 25, 2017 (JP) 2017-184099

(51) Int. Cl. A61F 2/82; A61F 2/958

(52) U.S. Cl. CPC A61F 2/82; A61F 2/958

(57) ABSTRACT

A treatment method includes performing a contrast inspection to observe a state of a body lumen, and temporarily forming an embolic site in the body lumen during the contrast inspection to temporarily bring the body lumen into an ischemic condition.

### CROSS-REFERENCE TO RELATED APPLICATION

This application claims priority to Japanese Patent Application No. 2017-184099 filed on Sep. 25, 2017, the entire content of which is incorporated herein by reference.

### TECHNICAL FIELD

The present invention relates to a treatment method for performing pre-conditioning to temporarily bring a body lumen into an ischemic condition.

### BACKGROUND

When required for a patient who has been admitted to a medical institution such as a hospital, a surgical operation, a procedure using a catheter, or the like may be performed. During the procedure, patients are temporarily in an ischemic condition, and may even be in a serious condition depending on the patient status. For patients who have relatively low tolerance to the ischemic condition, it is preferable that the patient be pre-treated to improve ischemic tolerance prior to the surgical operation or the procedure using a catheter. However, such pre-treatment has not yet been put to practical use in the current medical field. Such pre-treatment is called pre-conditioning. The pre-conditioning is a treatment that intentionally causes a blood vessel or the like of a patient to undergo an ischemic condition for a short period of time, thereby improving the ischemic tolerance.

As a technique relating to the pre-conditioning, for example, a method of using an inverse thermosensitive polymer is known (see, for example, U.S. Pat. No. 7,700,086). In this method, the inverse thermosensitive polymer is introduced into a body lumen to temporarily form an ischemic condition.

Regarding the pre-conditioning, there are experimental examples with mice and rats, but clinical validation for a human body is minimal at the present stage and thus clinical applications for the human body are desired. Clinical applications for the human body can be problematic, however, unless the burden or stress on a patient subjected to the pre-conditioning can be reduced and convenience can be improved.

### SUMMARY

The disclosure herein provides a treatment method in which the stress on a patient subjected to pre-conditioning is reduced and convenience is improved.

A treatment method according to the disclosure performs a contrast inspection for observing a state of a body lumen, and temporarily forming an embolic site in the body lumen during the contrast inspection in order to temporarily bring the body lumen into an ischemic condition.

In the treatment method according to the disclosure, the body lumen is temporarily brought into the ischemic condition by temporarily forming an embolic site in the body lumen during the contrast inspection for observing the state of the body lumen. Thus, when a surgeon such as a doctor determines that a patient is suffering from a severe disease (for example, myocardial infarction) by the contrast inspection, it is possible to improve the ischemic tolerance of the body lumen by temporarily forming the embolic site in the body lumen of the patient prior to performing the therapy procedure or surgery. Even if it is determined that the patient does not suffer from any serious disease, it is possible to improve the ischemic tolerance of the body lumen in the same manner as described above by temporarily forming the embolic site in the body lumen during the contrast inspection. Therefore, it is unnecessary for a subject such as a patient to visit a medical institution or the like in order to be subjected to pre-conditioning, as it is unnecessary to perform such treatment. Accordingly, the treatment method according to the disclosure can reduce the burden on the subject such as a patient who is subjected to pre-conditioning, and can improve the convenience of the pre-conditioning.

### BRIEF DESCRIPTION OF THE DRAWINGS

FIG. 1 is a block diagram illustrating a configuration of a medical system according to an exemplary embodiment of the disclosure;

FIG. 2 is a view illustrating a first introduction unit (injector) constituting the medical system illustrated in FIG. 1;

FIG. 3 is a view illustrating a second introduction unit (contrast catheter) constituting the medical system illustrated in FIG. 1;

FIG. 4 is a view illustrating a state where a stent is attached to a balloon of a balloon catheter;

FIG. 5 is an enlarged view of the balloon (expansion portion) in the balloon catheter illustrated in FIG. 4;

FIG. 6 is a flowchart illustrating a treatment method according to an exemplary embodiment of the disclosure;

FIG. 7 is a view for describing when a puncture site is formed in a living body and a catheter or the like is introduced;

FIG. 8 is a view illustrating an aspect of advancing the catheter that has been introduced into the living body to a coronary artery;

FIG. 9 is a view illustrating the treatment method according to the exemplary embodiment of the disclosure, and illustrates an aspect of discharging an embolic substance from an opening of the second introduction unit into a body lumen;

FIG. 10 is a view illustrating the treatment method according to the exemplary embodiment of the disclosure, and illustrates a state where an embolic site is formed in the body lumen by the second introduction unit;

FIG. 11 is a view illustrating the treatment method according to the exemplary embodiment of the disclosure, and illustrates a state where the embolic site that has been temporarily formed in the body lumen is dissolved;

FIG. 12 is a view illustrating an aspect of using the balloon catheter to expand a stenosis, and illustrates a state before expansion of the balloon;

FIG. 13 is a view illustrating an aspect of using the balloon catheter to expand the stenosis, and illustrates a state after expansion of the balloon; and

FIG. 14 is a view illustrating a state where the opening of the second introduction unit is arranged in a descending aorta near the coronary artery.

### DETAILED DESCRIPTION

Hereinafter, an exemplary embodiment of the disclosure will be described with reference to the accompanying drawings. Incidentally, the following description does not limit the technical scope or the meaning of terms described in the claims. In addition, dimensional ratios of the drawings are exaggerated for the convenience of description and may differ from actual ratios in some cases.

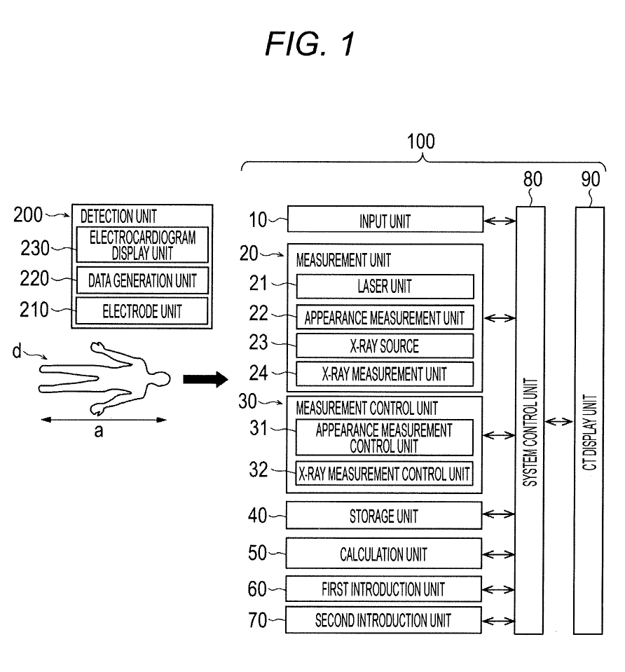

FIGS. 1 to 3 are drawings illustrating a medical system 1 used in a treatment method according to an exemplary embodiment of the disclosure.

When briefly described with reference to FIG. 1, the medical system 1 according to the illustrated exemplary embodiment includes an inspection unit 100 used for a contrast inspection of a subject (person to be inspected) d and a detection unit 200 used to confirm a blood flow state of the subject d. Details will be described hereinafter.

### Inspection Unit

As illustrated in FIG. 1, the inspection unit 100 includes: an input unit 10 configured to input information on the subject d; a measurement unit 20 configured to perform measurement on the subject d when generating a captured image of the subject d; a measurement control unit 30 configured to control the measurement unit 20; a storage unit 40 configured to store information necessary for generation of the captured image; a calculation unit 50 configured to perform calculations necessary for generation of the captured image; a first introduction unit 60 capable of introducing a contrast medium necessary for generation of the captured image into the subject d; a second introduction unit 70 capable of introducing an embolic substance to form an embolic site into the subject d; a system control unit 80 configured to control the inspection unit 100; and a CT display unit 90 configured to display the captured image and a contrast image generated by the calculation unit 50.

### Input Unit

The input unit 10 inputs data such as a medical record of a patient who is subjected to a contrast inspection. For example, data such as sex, age, drugs being taken, and past history of the subject d is input to the input unit 10.

### Measurement Unit

As illustrated in FIG. 1, the measurement unit 20 includes: a laser unit 21 which irradiates the subject d with a laser; an appearance measurement unit 22 which measures an appearance of the subject d; an X-ray source 23 which irradiates the subject d with X-rays; and an X-ray measurement unit 24 which measures the X-rays transmitted through the subject d from the X-ray source 23.

The laser unit 21 is constituted by a known oscillator, a mirror, and the like. The appearance measurement unit 22 has a sensor or the like that receives reflected light of the laser emitted from the laser unit 21. The X-ray source 23 forms X-rays in a shape such as a cone beam and emits the X-rays to the subject d. The X-ray measurement unit 24 measures the X-rays that have transmitted through the subject d.

### Measurement Control Unit

As illustrated in FIG. 1, the measurement control unit 30 includes: an appearance measurement control unit 31 which controls the appearance measurement unit 22; and an X-ray measurement control unit 32 which controls the X-ray measurement unit 24.

The appearance measurement control unit 31 performs laser output control, position control, position control of the subject d, timing control, and the like with respect to the appearance measurement unit 22. The X-ray measurement control unit 32 performs X-ray output control, various types of position control for outputting X-ray beams, timing control, and the like.

### Storage Unit

The storage unit 40 stores data input to the input unit 10, data measured by the measurement unit 20, captured image data generated by the calculation unit 50, and the like.

### Calculation Unit

The calculation unit 50 generates an appearance captured image from the data measured by the appearance measurement unit 22 and generates an X-ray CT captured image from the data measured by the X-ray measurement unit 24.

### First Introduction Unit

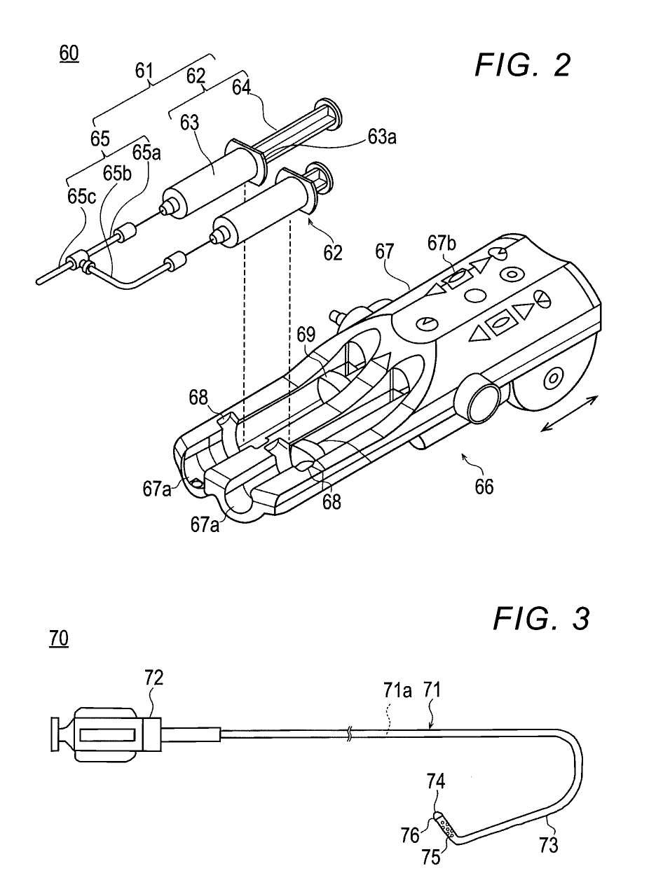

The first introduction unit 60 is used for introduction of the contrast medium into the body lumen. As illustrated in FIG. 2, the first introduction unit 60 has an injection unit 61 which can be filled with the contrast medium or the like, and an operation unit 66 used for an injection operation by the injection unit 61.

As illustrated in FIG. 2, the injection unit 61 includes a plurality of syringes 62 used for injection of the contrast medium and the like, and a connection tube 65 which connects the plurality of syringes 62 to each other. The syringe 62 includes: a cylinder 63 having an accommodating space to accommodate the contrast medium and the like; and a piston 64 which discharges the contrast medium or the like accommodated in the accommodation space inside the cylinder 63 to the outside.

As illustrated in FIG. 2, the cylinder 63 has a hollow cylindrical shape. A flange 63a is provided at an axial end of the cylindrical shape of the cylinder 63. The piston 64 is configured to be movable in the accommodating space of the cylinder 63. As illustrated in FIG. 2, the two syringes 62 are provided in the exemplary embodiment. However, the number of syringes is not limited to two as long as the syringe can accommodate the contrast medium and the embolic substance to be described later.

As illustrated in FIG. 2, the connection tube 65 includes: branch tubes 65a and 65b connected to the syringes 62; and a junction tube 65c which is continuous from the branch tubes 65a and 65b and formed such that tubes of the branch tubes 65a and 65b are integrated into one tube.

The branch tubes 65a and 65b are provided in accordance with the number of the syringes 62 of the first introduction unit 60. The junction tube 65c is configured such that a lumen thereof communicates with lumens of the branch tubes 65a and 65b so that the contrast medium and the embolic substance discharged from the syringe 62 can flow therethrough.

As illustrated in FIG. 2, the operation unit 66 includes: a housing 67 provided with, for each syringe 62, an installation portion (a recess 67a) in which the syringe 62 can be installed and an arrangement portion 67b in which operation buttons configured to perform various operations are arranged; a syringe adapter 68 configured to attach the syringe 62 to the housing 67; and a drive mechanism 69 that applies a driving force to discharge the contrast medium or the like accommodated in the accommodating space of the cylinder 63 to the outside.

The recess 67a is formed so as to substantially match an outer shape of the syringe 62. As illustrated in FIG. 2, the recess 67a is formed so as to extend along the longitudinal direction of the housing 67. The arrangement portion 67b is provided to be adjacent to a rear end side of the recess 67a at the time of use.

The syringe adapter 68 fixes a position of the syringe 62 to the recess 67a. The syringe adapter 68 is installed in the recess 67a of the housing 67 and configured to be engaged with the flange 63a of the syringe 62 in a state where the syringe 62 is arranged in the recess 67a. However, the specific structure or the like of the syringe adapter 68 is not particularly limited as long as the syringe adapter 68 can secure the syringe 62 in position with respect to the housing 67. For example, a groove shape having the same function as the syringe adapter 68 may be provided in the recess 67a of the housing 67.

The drive mechanism 69 is constituted by a drive motor (not illustrated) and a gear pair (not illustrated) that converts rotational motion of the motor into linear motion. The drive mechanism 69 is configured to be capable of applying a driving force to the piston 64 in the state where the syringe 62 is installed in the recess 67a. As a result, the piston 64 moves inside the accommodating space of the cylinder 63, and the contrast medium accommodated in the accommodating space of the cylinder 63 is delivered via the connection tube 65 by mechanical driving. Incidentally, the first introduction unit 60 can be used for introduction of not only the contrast medium but also the embolic substance as will be described later.

### Second Introduction Unit

The second introduction unit 70 is used at the time of introducing the embolic substance to form the embolic site into the body lumen. As illustrated in FIG. 3, the second introduction unit 70 includes: a main body portion 71 having flexibility; and a hub 72 attached to a proximal end of the main body portion 71.

The main body portion 71 is configured using an elongated tubular member. The main body portion 71 is formed in a substantially linear shape. As illustrated in FIG. 3, the main body portion 71 has a deformable portion 73 formed by bending from a substantially linear shape on a proximal end side in a state (natural state) where no external force is applied on a distal end side.

As illustrated in FIG. 3, the main body portion 71 has a lumen 71a through which the contrast medium or the like flows from the proximal end side to the distal end side. A distal end opening 74 is provided at a distal end of the deformable portion 73 corresponding to the distal end of the main body portion 71. An X-ray opaque marker (not shown) may be provided at the distal end opening 74. In addition, a plurality of side holes 75 is formed in an outer surface of the deformable portion 73. The side hole 75 communicates with the lumen 71a formed from the proximal end side to the distal end side. A soft tip 76 is provided at a distal end of the deformable portion 73 of the main body portion 71. In the illustrated embodiment, the distal end opening 74 and the side hole 75 correspond to openings.

The hub 72 is used to inject a liquid such as the contrast medium or a medicinal liquid.

Examples of a constituent material of the main body portion 71 include polyolefins such as polyethylene, polypropylene, and an ethylene-vinyl acetate copolymer, a polyamide resin (for example, nylon 11, nylon 12, and nylon 6), a polyester-polyamide resin (for example, product name: Grilux, manufactured by DIC Corporation), a polyether-polyamide resin (for example, product name: Pebax, manufactured by Arkema Group), polyurethane, an ABS resin, an AS resin, a fluorine resin (PFA, PTFE, ETFE, and the like), polyimide, a shape-memory resin, a thermoplastic elastomer of a polyester type, a polyamide type, a polyurethane type, and the like, or various synthetic resins containing the above-described materials such as a polymer blend and a polymer alloy.

The insertion of the second introduction unit 70 into the living body is performed while confirming its position under X-ray fluoroscopy. Thus, it is preferable to blend an X-ray opaque material such as barium sulfate, bismuth oxide, and tungsten into the material forming the main body portion 71.

The type of contrast medium is not particularly limited, but a material having X-ray opacity such as barium sulfate can be used. In addition, a material of the catheter is not particularly limited, but examples thereof can include polyethylene and the like.

An outer diameter of the main body portion 71 is not particularly limited, but is preferably about 2.7 mm or smaller, and more preferably about 2.0 mm or smaller, in general.

In addition, an inner diameter of the main body portion 71 is not particularly limited, but is preferably about 0.9 to 1.8 mm, and more preferably about 1.0 to 1.5 mm, in general.

### Embolic Substance

In the treatment method according to the exemplary embodiment to be described later, the embolic substance is introduced into the body lumen using the first introduction unit 60 or the second introduction unit 70 in order to form an ischemic site in the body lumen. The embolic substance that has been introduced into the body lumen temporarily remains in the body lumen, thereby forming the embolic site temporarily in the body lumen.

In the exemplary embodiment, examples of the embolic substance can include an inverse thermosensitive polymer containing poloxamer or poloxamine and transitioning from a liquid or a soft gel to a hard gel depending on body temperature, an embolic material containing micro-starch and mitomycin C, and the like. The micro-starch and the mitomycin C can be formed in a granular shape and can be introduced into the living body by being mixed with a saline solution as will be described later. Sizes (particle sizes) of the micro-starch and mitomycin C are not particularly limited, and can be 100 to 300 μm, for example.

### System Control Unit, CT Display Unit

The system control unit 80 controls the operation of the entire system in the inspection unit 100. The CT display unit 90 is configured using a monitor or the like which displays a CT image and the like generated by the input unit 10 and the calculation unit 50.

### Detection Unit

The detection unit 200 is used to confirm whether the body lumen has reached an ischemic condition due to the introduction of the embolic substance. The detection unit 200 is configured using a known electrocardiogram or the like in the exemplary embodiment. As illustrated in FIG. 1, the detection unit 200 includes an electrode unit 210, a data generation unit 220, and an electrocardiogram display unit 230.

The electrode unit 210 includes an electrode to be attached to a body surface or the like of the subject d and detects an electrical signal of the heart. The data generation unit 220 generates electrocardiogram data based on the electrical signal detected by the electrode unit 210. The electrocardiogram display unit 230 is configured using a known monitor or the like, and displays a waveform of the electrocardiogram generated by the data generation unit 220 and the like.

### Treatment Method

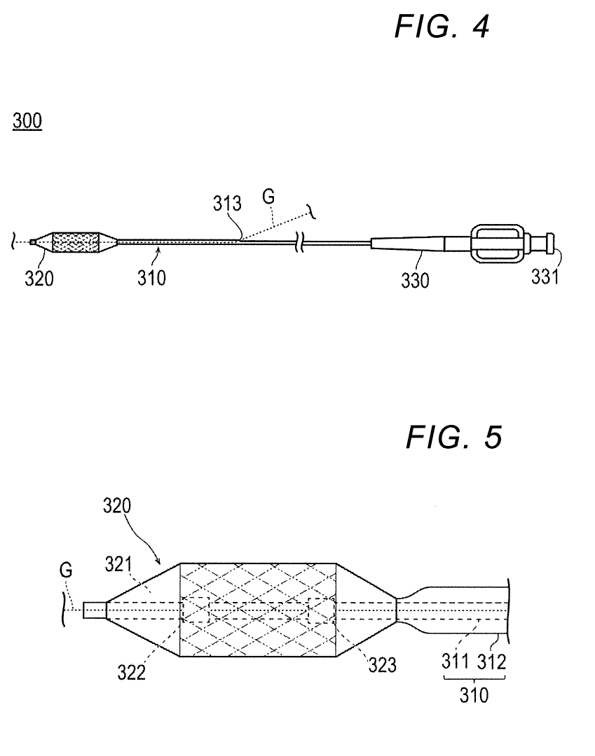

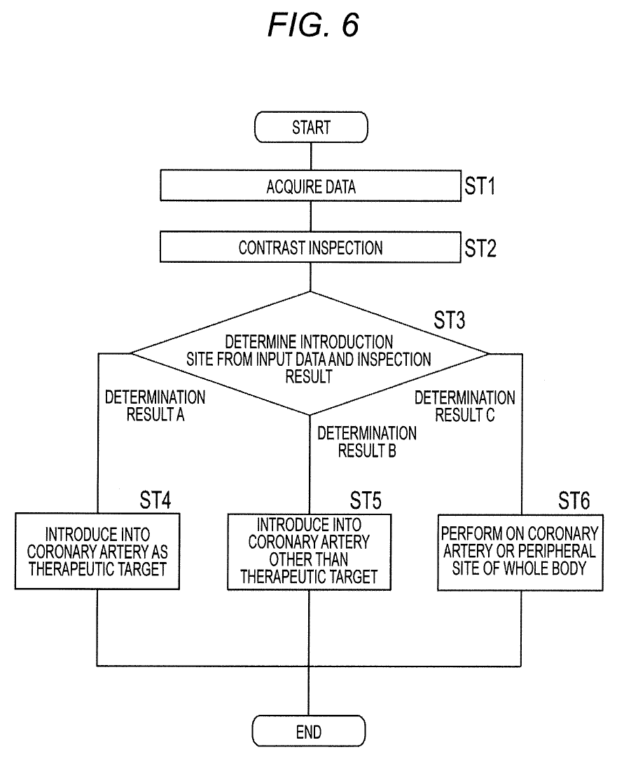

The treatment method using the medical system 1 according to the exemplary embodiment and a therapeutic method that is performed after the treatment method will be described with reference to FIGS. 4 to 13. FIGS. 6 to 11 are drawings for describing the treatment method using the medical system 1 according to the exemplary embodiment. FIGS. 4, 5, 12, and 13 are drawings for describing the therapeutic method that is performed after the treatment method. In the exemplary embodiment, the contrast inspection of the treatment method will be described relative to a case of performing coronary angiography (CAG) in the coronary artery.

As briefly described with reference to FIG. 6, the treatment method according to the exemplary embodiment includes: a step (ST1) of acquiring information on the subject d from a medical chart or the like; a step (ST2) of generating a CT captured image of the subject d by using the inspection unit 100 and performing a contrast inspection using the CT captured image; and steps (ST3 to ST6) of forming an embolic site R in any body lumen Bl of the subject d from the input data of the subject d and a result of the contrast inspection. Details will be described hereinafter.

First, the input unit 10 acquires data such as a medical chart of the subject d or the like (ST1). The data of the subject d using the input unit 10 may be acquired by inputting data recorded on a paper medium from a computer or the like, through manual input by a medical staff, or by acquiring data of an electronic medical chart via a network.

Next, a contrast medium is introduced into a living body of the subject d by a surgeon in order to acquire the CT captured image of the subject d.

During the introduction of the contrast medium, a brachial vein of the body lumen Bl is punctured using a puncture needle (not illustrated) or the like to form a puncture site of the first introduction unit 60.

Next, the first introduction unit 60 is pierced into the puncture site formed in the above-described manner. Next, a button provided in the operation unit 66 is pressed down. As a result, the drive mechanism 69 is actuated so that the piston 64 is moved by mechanical driving, and the contrast medium is discharged from the accommodating space of the cylinder 63 constituting the syringe 62 and introduced into the body lumen Bl.

Next, the measurement unit 20 and the like are actuated to generate a body image in the coronary artery. Specifically, a height position of the subject d (position of the subject d in a direction orthogonal to the plane of a captured image (see arrow a in FIG. 1)) to generate the CT captured image is determined by the laser unit 21 and the appearance measurement unit 22. Next, X-ray irradiation, image processing, and the like are performed using the X-ray source 23 and the X-ray measurement unit 24 to generate the CT captured image. The generated captured image is displayed on the CT display unit 90 (ST2).

Next, the surgeon determines an introduction site of an embolic substance M while referring to the input data of the subject d and the CT captured image (ST3).

When a lesion such as a stenosis has been found in the coronary artery of the subject d from the input data of the subject d and the CT captured image so that therapy for the stenosis is required, and it has been determined that there is no problem if the embolic substance M is introduced into the coronary artery which is a therapeutic site (ST3 in FIG. 6: Determination Result A), the embolic substance M is introduced into the coronary artery as the therapeutic site (ST4). The system control unit 80 determines the introduction site.

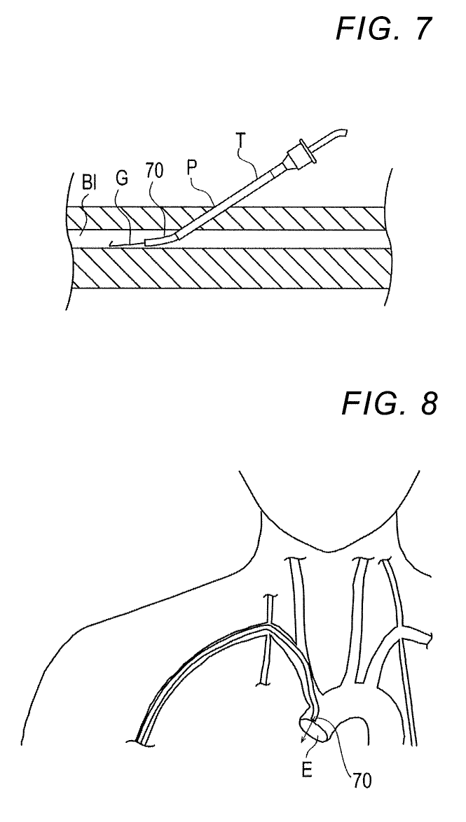

Specifically, an upper arm or a radial artery of the body lumen Bl is punctured using a puncture needle (not illustrated) or the like to form a puncture site P of the second introduction unit 70 as illustrated in FIG. 7.

Next, an introducer T is inserted through the puncture site P to secure an introduction port of a guide wire G as illustrated in FIG. 7. The guide wire G is introduced into a lumen of the introducer T and introduced to the coronary artery which is a target site. A marker has been attached to a distal end of the guide wire G so that it is possible to confirm a position of the guide wire G under X-ray radiography.

When the guide wire G reaches the target site, a contrast catheter, which is the second introduction unit 70, is introduced along the guide wire G from the puncture site P into the body lumen Bl.

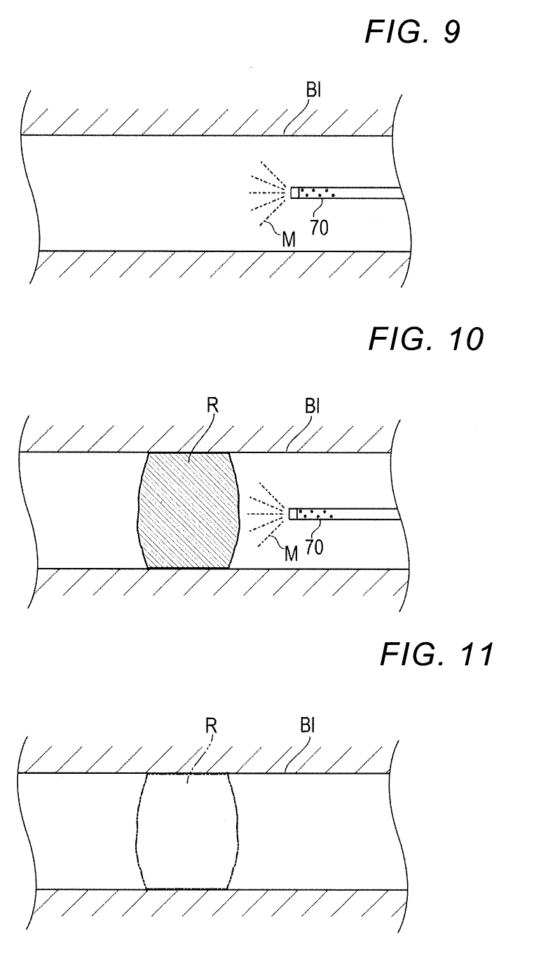

The distal end opening 74 of the second introduction unit 70 is moved to the coronary artery positioned at a distal end of an ascending aorta E illustrated in FIG. 8 while confirming the CT captured image. Then, the embolic substance M is mixed with a saline solution or the like, and the mixture is caused to flow from the proximal end side to the distal end side of the second introduction unit 70 and is discharged from the distal end opening 74 as illustrated in FIG. 9.

As a result, the embolic site R is temporarily formed in the body lumen Bl (coronary artery) into which the embolic substance M has been introduced as illustrated in FIG. 10. As the embolic site R is formed, the body lumen Bl is temporarily brought into an ischemic condition. The ischemic condition can be confirmed by recognizing the ST-segment elevation in a waveform of the detection unit 200 (electrocardiogram).

As illustrated in FIG. 11, the embolic substance M is preferably dissolved in 10 to 15 minutes (in about 30 minutes at most). As a result, the subject d (patient) can acquire ischemia tolerance or be enhanced in ischemia tolerance by introducing the embolic substance M.

### Therapeutic Method

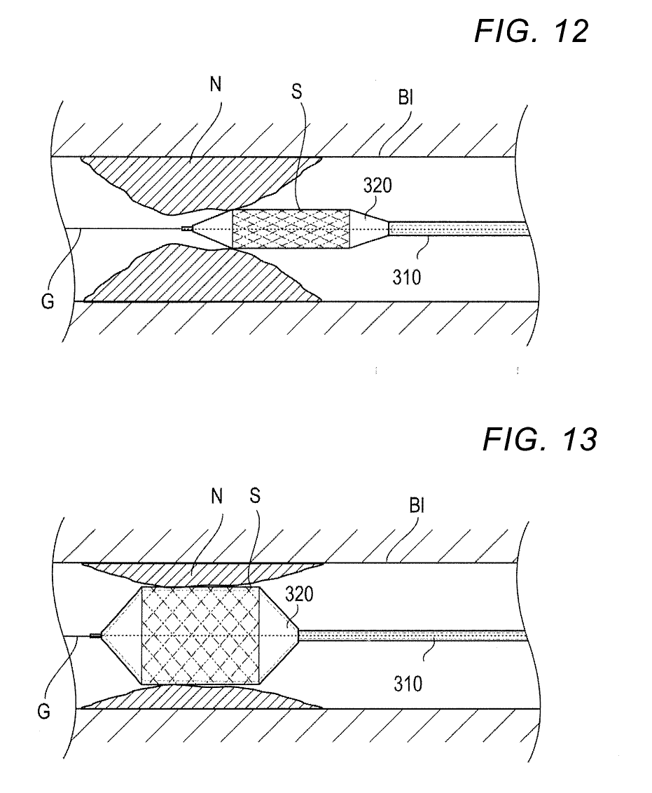

Next, the therapeutic method will be described. A therapy is performed on the therapeutic site (lesion) when a predetermined time has elapsed after the embolic substance in the body lumen Bl dissolves (for example, within 48 hours). When the lesion is, for example, a stenosis N that has formed in the body lumen Bl as illustrated in FIG. 12, a known balloon catheter 300 (corresponding to an elongated medical instrument) illustrated in FIGS. 4 and 5 is used to expand the stenosis N.

### Balloon Catheter

As illustrated in FIG. 4, the balloon catheter 300 includes: a shaft portion 310 which is an elongated tubular member; a balloon 320 (corresponding to an expansion portion) provided at a distal end of the shaft portion 310; and a hub 330 provided at a proximal end of the shaft portion 310.

The shaft portion 310 is formed using a hollow tubular member. As illustrated in FIG. 5, the shaft portion 310 includes: an inner tube 311 provided with a guide wire lumen through which the guide wire G is inserted; and an outer tube 312 having an expansion lumen which communicates with a lumen 321 of the balloon 320 and through which a fluid for expanding the balloon 320 can flow.

As illustrated in FIG. 4, an introduction hole 313 configured to allow the guide wire G to be inserted into the shaft portion 310 is provided at an intermediate portion of the shaft portion 310 in the longitudinal direction. As illustrated in FIG. 5, contrast markers 322 and 323 are provided in the lumen 321 of the balloon 320. A proximal end opening 331 is provided at a proximal end of the hub 330, and it is configured such that the expansion fluid for expanding the balloon 320 can be supplied from the proximal end opening 331.

The balloon catheter 300 forms a puncture site at a site in the vicinity of a site where the first introduction unit 60 has been introduced. Then, the introducer T is inserted and the guide wire G is inserted into the lumen of the introducer T from the puncture site in the same manner as during the treatment method. Then, the balloon catheter 300 is introduced from the lumen of the introducer T into the body lumen Bl and advanced along the guide wire G to the therapeutic site.

As illustrated in FIG. 12, when the balloon 320 of the balloon catheter 300 reaches the target site, the balloon 320 is expanded by an inflator (not illustrated). As a result, a stent S expands in accordance with the expansion of the balloon 320 to expand the stenosis N of the body lumen Bl as illustrated in FIG. 13. When the stenosis N is sufficiently expanded, the balloon 320 is contracted, the balloon catheter 300 is removed from the body lumen Bl in a state where the stent S indwells, and the guide wire G is removed.

With respect to the treatment method, different from Step ST4 illustrated in FIG. 6, when the therapy for the stenosis of the coronary artery is required, the degree of ischemia of the stenosis is serious, and it has been determined that it is difficult to introduce the embolic substance into the stenosis as the therapeutic site (ST3 in FIG. 6: Determination Result B), the embolic substance M is introduced into the coronary artery other than the therapeutic site (ST5). Specifically, the position of the distal end opening 74 of the second introduction unit 70 is moved from the therapeutic site to the distal end side or proximal end side while observing the CT image. Then, the embolic substance M is discharged from the distal end opening 74 and the side hole 75 of the second introduction unit 70 in the same manner as described above. Thereafter, a therapy for the lesion such as the above-described expansion of the stenosis is performed after a lapse of a predetermined time (see FIGS. 12 and 13).

Different from Steps ST4 and ST5 illustrated in FIG. 6, when it has been determined that the therapy for the lesion part is unnecessary (ST3 in FIG. 6: Determination Result C), only the introduction of the embolic substance M is performed on a peripheral site of the whole body (ST6).

Specifically, the above-described upper arm or radial artery is punctured using a puncture needle (not illustrated) or the like to form a puncture site. Next, in a state where the cylinder 63 is filled with the embolic substance M, the first introduction unit 60 is pierced into the puncture site in the same manner as the time of introducing the contrast medium. Next, the drive mechanism 69 is actuated by pressing down the button provided on the operation unit 66. As a result, the embolic substance M filling the cylinder 63 is introduced into the body lumen Bl via the connection tube 65.

Since the driving force is applied by the motor or the like, the first introduction unit 60 can introduce the embolic substance M to the peripheral site of the living body which is more distant from a discharge site as compared to the second introduction unit 70. A discharge amount of the embolic substance M can be appropriately adjusted by operating the button provided on the operation unit 66.

After the introduction of the embolic substance M is completed, the first introduction unit 60 is removed from the body lumen Bl.

As described above, in the treatment method according to the exemplary embodiment, it is configured such that the contrast inspection for observing the state of the body lumen Bl is performed, and the embolic site R is temporarily formed in the body lumen Bl during the contrast inspection to temporarily bring the body lumen Bl into the ischemic condition.

As a result, even when it is determined that the subject d is suffering from a serious disease, it is possible to improve the ischemic tolerance by forming the embolic site R in the body lumen Bl during the contrast inspection. In addition, even if the subject d is not suffering from any serious disease, it is possible to perform preventive treatment (temporary formation of the embolic site R) against a serious symptom that may appear after the exercise during the inspection. As the embolic site R is formed in the body lumen Bl during the inspection in this manner, it is possible to eliminate the need of visiting a medical facility or the like solely for improving the ischemic tolerance. Accordingly, it is possible to reduce a burden on the patient who is required to form the embolic site R (pre-conditioning) and to improve convenience.

In addition, the formation of the embolic site R is performed in the state where the embolic substance M that dissolves over time has been introduced into the body lumen Bl. Thus, the burden on the patient can be reduced as compared with the case where the formation of the embolic site R is performed by a procedure.

Further, the introduction of the embolic substance M can be performed after the contrast inspection. As a result, it is possible to introduce the embolic substance M into a more appropriate site upon grasping the ischemic condition of the subject d from the result of the contrast inspection.

The introduction of the embolic substance M can be performed in the state where the distal end opening 74 and the side hole 75 of the second introduction unit 70, which is the elongated tubular member that has been inserted into the body lumen Bl through the puncture site P formed in the body surface, are delivered to the target site such as the coronary artery. Thus, the embolic substance M can be discharged to the target site in the state where the tubular member of the second introduction unit 70 is arranged in the vicinity of the target site, and the ischemic condition with respect to the subject d can be precisely controlled.

According to a further aspect of the invention, the introduction of the embolic substance M can be performed by causing the embolic substance M, which has been delivered by mechanical driving of the motor or the like, such as the drive mechanism 69 of the first introduction unit 60, to flow into the body lumen Bl through the puncture site formed in the body surface to be delivered to the target site. As a result, it is possible to introduce the embolic substance M to the peripheral side which is more distant from the distal end of the medical instrument as compared to a case where the embolic substance M is manually introduced from a medical instrument such as a catheter. As a result, it is possible to promptly bring the peripheral site into the ischemic condition.

In addition, it is possible to perform the procedure of introducing the elongated balloon catheter 300 having the expandable balloon 320 into the body lumen Bl and expanding the stenosis N using the balloon 320 after the embolic substance M arranged in the body lumen Bl has dissolved and in the state where the stenosis N has been formed at a site identical to or different from the site at which the embolic substance M is arranged. Since the embolic substance M is introduced into any site of the body lumen Bl before the procedure using the balloon catheter 300, it is possible to perform the necessary therapy on the stenosis N in the state where the ischemic tolerance of the subject d has been improved.

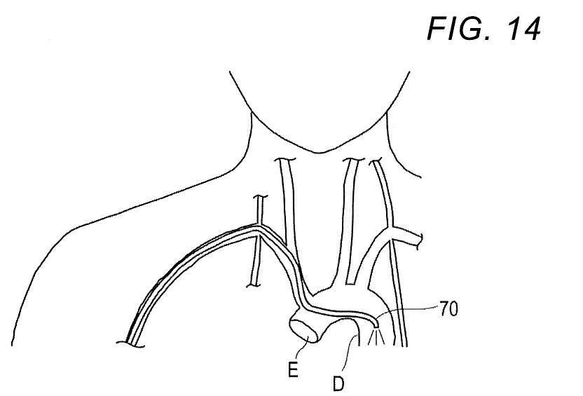

It should be noted that the invention is not limited to the above-described embodiment, and various modifications can be made within the scope of the claims. In the above description, the exemplary embodiment in which the distal end opening 74 and the side hole 75 of the tubular member, which is the second introduction unit 70, are introduced in the vicinity of the coronary artery has been described. However, the disclosure is not limited thereto, and the distal end opening 74 and the side hole 75 of the second introduction unit 70 may be arranged so as to face a lower limb side in a descending aorta D as illustrated in FIG. 14 other than the above-described manner. As a result, when the embolic substance M is discharged, the embolic substance M is discharged to an organ on the lower limb side of the descending aorta and the like, and it is possible to prevent the embolic substance M from being discharged to a cerebral blood vessel.

In addition, the exemplary embodiment in which the contrast inspection for the subject d is performed on the coronary artery of the subject d such as the CAG has been described as illustrated in FIG. 6 in the above description, but the disclosure is not limited thereto. In addition to the above embodiment, it may be configured such that the inspection unit 100 generates the CT captured image for the whole body of the subject d.

In addition, the introduction of the embolic substance M is performed after the contrast inspection in the above description, but the disclosure is not limited thereto. When a site of the subject d (patient) that requires a therapy is predicted, the introduction of the embolic substance M may be performed before the contrast inspection or at the same time as the contrast inspection as long as the embolic substance M is introduced to a site other than a therapy-planned site.

In addition, the exemplary embodiment in which the detection unit 200 is configured using the electrocardiogram and the ischemic condition of the body lumen Bl of the subject d is confirmed by the electrocardiogram waveform has been described in the above description. However, the disclosure is not limited thereto, and the ischemic condition may be determined by a fractional flow ratio (FFR).

The fractional flow ratio is calculated by inserting the elongated tubular member provided with a pressure sensor at a distal end, which is a pressure wire, into the coronary artery while instilling a coronary artery dilator and measuring a pressure of each part. In the pressure measurement, a pressure (Pa) on the proximal side of the stenosis in the coronary artery and a pressure (Pd) on the distal side are measured, and the FFR is calculated by Pd/Pa. When the FFR having a value lower than 0.75 is continuously obtained, it is possible to determine that the ischemic site is formed in the body lumen by the embolic substance M.

In addition, the exemplary embodiment in which the embolic site R is formed by introducing the embolic substance M into the body lumen Bl has been described in the above description, but the disclosure is not limited thereto. In addition to the above exemplary embodiment, the balloon catheter 300 may be introduced into the body lumen Bl of the coronary artery or the like during the contrast inspection, the embolic site R may be formed by expanding the balloon 320, and the body lumen Bl may be temporarily brought into the ischemic condition.

Although the introduction site of the embolic substance M is determined by the system control unit 80 in the above-described treatment method, the disclosure is not limited thereto. The surgeon such as a doctor may determine the introduction site other than the above-described manner.

The detailed description above describes features, characteristics and operational aspects of embodiments of a treatment method representing examples of the treatment method disclosed herein. The disclosure and the present invention are not limited, however, to the precise embodiments and variations described. Various changes, modifications and equivalents could be effected by one skilled in the art without departing from the spirit and scope of the disclosure as defined in the appended claims. It is expressly intended that all such changes, modifications and equivalents which fall within the scope of the claims are embraced by the claims.

What is claimed is:

1. A treatment method for temporarily bringing a body lumen into an ischemic condition; the method comprising:
performing a contrast inspection on a state of the body lumen; and
temporarily forming an embolic site in the body lumen during the contrast inspection and thereby temporarily bringing the body lumen into an ischemic condition.

2. The treatment method according to claim 1, wherein temporarily forming the embolic site includes introducing an embolic substance that dissolves over time into the body lumen.

3. The treatment method according to claim 2, wherein introducing the embolic substance is performed after performing the contrast inspection.

4. The treatment method according to claim 2, wherein introducing the embolic substance includes introducing the embolic substance via an opening of an elongated tubular member, the embolic substance being delivered to a predetermined site.

5. The treatment method according to claim 4, further comprising inserting the elongated tubular member via a puncture site formed in a body surface.

6. The treatment method according to claim 4, wherein the predetermined site is a descending aorta.

7. The treatment method according to claim 2, wherein introducing the embolic substance includes causing the embolic substance to flow into the body lumen through a puncture site formed in a body surface to be delivered to a target site.

8. The treatment method according to claim 7, wherein introducing the embolic substance further includes delivering the embolic substance by mechanical driving.

9. The treatment method according to claim 2, further comprising:
introducing an elongated medical instrument having an expandable expansion portion into the body lumen after the embolic substance dissolves, the body lumen having a stenosis formed at a site identical to or different from a site at which the embolic substance is introduced; and
expanding the stenosis using the expansion portion.

10. The treatment method according to claim 2, wherein introducing the embolic substance includes providing an introduction unit comprising an elongated tubular member.

11. The treatment method according to claim 10, wherein the elongated tubular member includes a distal end opening and a plurality of side holes,

12. The treatment method according to claim 2, wherein introducing the embolic substance include providing an introduction unit comprising at least one syringe.
